(19) United States
(12) Patent Application Publication
(10) Pub. No.: US 2008/0261958 A1
(43) Pub. Date: Oct. 23, 2008

(54) Combination of Organic Compounds

(76) Inventor: Randy Lee Webb, Flemington, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/092,455

(22) PCT Filed: Nov. 6, 2006
(86) PCT No.: PCT/US2006/043250

§ 371 Date: May 2, 2008

Related U.S. Application Data
(60) Provisional application No. 60/734,700, filed on Nov. 8, 2005.

(51) Int. Cl. A61K 31/546; A61K 31/41; A61K 31/445; A61P 9/00; A61K 31/165

(52) U.S. Cl. 514/222.5; 514/381; 514/319; 514/620

(57) ABSTRACT

The present invention relates to a combination comprising: (a) an angiotensin II receptor blocker (ARB), or a pharmaceutically acceptable salt thereof; (b) a calcium channel blocker (CCB), or a pharmaceutically acceptable salt thereof; and (c) one of the two active agents selected from (i) a rennin inhibitor, or a pharmaceutically acceptable salt thereof; and (ii) a neutral endopeptidase (NEP) inhibitor, or a pharmaceutically acceptable salt thereof; for the prevention of, delay the onset of and/or treatment of cardiovascular disorders, which method comprises administering to a warm-blooded animal, in need thereof, a therapeutically effective amount of a combination of the present invention.

In one aspect, the present invention relates to a combination, such as a pharmaceutical combination, comprising:
(a) an angiotensin II receptor blocker (ARB), or a pharmaceutically acceptable salt thereof;
(b) a calcium channel blocker (CCB), or a pharmaceutically acceptable salt thereof; and
(c) one of the two active agents selected from
   (i) a renin inhibitor, or a pharmaceutically acceptable salt thereof; and
   (ii) a neutral endopeptidase (NEP) inhibitor, or a pharmaceutically acceptable salt thereof.

In a further aspect, the present invention provides a method for the prevention of, delay the onset of and/or treatment of cardiovascular disorders which method comprises administering to a warm-blooded animal, including man, in need thereof, a therapeutically effective amount of a combination comprising:
(a) an angiotensin II receptor blocker (ARB), or a pharmaceutically acceptable salt thereof;
(b) a calcium channel blocker (CCB), or a pharmaceutically acceptable salt thereof; and
(c) one of the two active agents-selected from
   (i) a renin inhibitor, or a pharmaceutically acceptable salt thereof; and
   (ii) a neutral endopeptidase (NEP) inhibitor, or a pharmaceutically acceptable salt thereof.

Cardiovascular disorders include, but are not limited to, hypertension (whether for malignant, essential, reno-vascular, diabetic, isolated systolic, or other secondary type of hypertension), heart failure such as diastolic and congestive heart failure (acute and chronic), left ventricular dysfunction, endothelial dysfunction, diastolic dysfunction, hypertrophic cardiomyopathy, diabetic cardiac myopathy, supraventricular and ventricular arrhythmias, atrial fibrillation (AF), cardiac fibrosis, atrial flutter, detrimental vascular remodeling, plaque stabilization, myocardial infarction (MI) and its sequelae, atherosclerosis including coronary arterial disease (CAD), angina pectoris (whether unstable or stable), renal insufficiency (diabetic and non-diabetic), renal fibrosis, polycystic kidney disease (PKD), type 2 diabetes, metabolic syndrome, secondary aldosteronism, primary and secondary pulmonary hypertension, renal failure conditions such as nephrotic syndrome, diabetic nephropathy, glomerulonephritis, scleroderma, glomerular sclerosis, proteinuria of primary renal disease, renal vascular hypertension, diabetic retinopathy and end-stage renal disease (ESRD), the management of other vascular disorders such as migraine, peripheral vascular disease (PVD), Raynaud's disease, luminal hyperplasia, cognitive dysfunction (such as Alzheimer's), glaucoma and cerebrovascular disease such as embolic or thrombotic stroke.

Prolonged and uncontrolled hypertensive vascular disease ultimately leads to a variety of pathological changes in target organs such as the heart and kidney. Furthermore, sustained hypertension may lead to an increased occurrence of stroke. Therefore, there has been a strong need to evaluate the efficacy of anti-hypertensive therapy by an examination of additional cardiovascular endpoints, beyond those of blood pressure lowering, to get further insight into the benefits of the treatment with anti-hypertensive agents.

The nature of hypertensive vascular diseases is multifactorial, and under certain circumstances, therapeutic agents with different mechanism of action have been combined. However, just considering any combination of drugs having different mode of action does not necessarily lead to drug combinations with advantageous effects. Accordingly, there is an urgent need to identify more efficacious therapies, in particular combination therapies, which have less deleterious side effects for the treatment of, e.g., cardiovascular and renal diseases as described herein above.

The natural enzyme renin released from the kidneys cleaves angiotensinogen in the circulation to form the decapeptide called angiotensin I. This in turn is cleaved by angiotensin converting enzyme (ACE) in the lungs, kidneys and other organs to form the octapeptide called angiotensin II. Through its interaction with specific receptors on the surface of the target cells the octapeptide increases blood pressure both directly by arterial vasoconstriction and indirectly by liberating from the adrenal glands the sodium-ion-retaining hormone aldosterone, accompanied by an increase in extracellular fluid volume. It has been possible to identify receptor subtypes that are termed, e.g., AT₁- and AT₂-receptors.

Inhibitors of the enzymatic activity of renin bring about a reduction in the formation of angiotensin I. As a result a smaller amount of angiotensin II is produced. The reduced concentration of that active peptide hormone is the direct cause of, e.g., the antihypertensive effect of renin inhibitors. Accordingly, renin inhibitors, or salts thereof, may be employed, e.g., as antihypertensives or for treating congestive heart failure.

On the other hand, in recent times great efforts have been made to identify substances that antagonize the AT₁-receptor. Such active ingredients are often called as angiotensin II antagonists or angiotensin II blockers (ARBs). As a result of the inhibition of the AT₁-receptor activity such antagonists may also be employed, e.g., as antihypertensives or for the treatment of congestive heart failure, among other indications. Angiotensin II blockers are therefore understood to be those active agents which bind to the AT₁-receptor subtype but do not result in activation of the receptor.

Further evaluations have revealed that renin inhibitors and angiotensin II blockers may also be employed for a much broader range of therapeutic indications.

Neutral endopeptidase (EC 3.4.24.11; enkephalinase; atriopeptidase; NEP; Biochem. J., 241, p. 237-247, 1987) is a zinc-containing metalloprotease that cleaves a variety of peptide substrates on the amino terminal side of aromatic amino acids. Substrates for this enzyme include, but are not limited to, atrial natriuretic factors (ANF, also known as ANP), brain natriuretic peptide (BNP), met and leu enkephalin, bradykinin, neurokinin A, and substance P.

ANPs are a family of vasodilator, diuretic and antihypertensive peptides which have been the subject of many recent reports in the literature, e.g., Annu. Rev. Pharm. Tox., 29, 23-54, 1989. One form, ANF 99-126, is a circulating peptide hormone which is released from the heart during conditions of cardiac distension. The function of ANF is to maintain salt and water homeostasis as well as to regulate blood pressure. ANF is rapidly inactivated in the circulation by at least two processes: by a receptor-mediated clearance as reported in Am. J. Physiol., 256, R469-R475, 1989, and by an enzymatic inactivation via NEP as described in Biochem. J., 243, 183-187, 1987. It has been previously demonstrated that inhibitors of NEP potentiate the hypotensive, diuretic, natriuretic and plasma ANF responses to pharmacological injection of ANF in experimental animals. The potentiation of ANF by two specific NEP inhibitors is reported by Sybertz et al. in J. Pharmacol. Exp. Ther. 250, 2, 624-631, 1989, and in Hypertension, 15, 2, 152-161, 1990, while the potentiation of ANF by NEP in general was disclosed in U.S. Pat. No. 4,749,688. In U.S. Pat. No. 4,740,499 Olins disclosed the use of thiorphan and kelatorphan to potentiate atrial peptides. Moreover, NEP inhibitors lower blood pressure and exert ANF-like effects such as diuresis and increased cyclic guanosine 3′,5′-monophosphate (cGMP) excretion in some forms of experimental hypertension. The antihypertensive action of NEP inhibitors is mediated through ANF because antibodies to ANF will neutralize the reduction in blood pressure.

Listed below are some of the definitions of various additional terms used herein to describe certain aspects of the present invention. However, the definitions used herein are those generally known in the art, e.g., hypertension, heart failure and atherosclerosis, and apply to the terms as they are used throughout the specification unless they are otherwise limited in specific instances.

The term “prevention” refers to prophylactic administration to healthy patients to prevent the development of the conditions mentioned herein. Moreover, the term “prevention” means prophylactic administration to patients being in a pre-stage of the conditions to be treated.

The term “delay the onset of”, as used herein, refers to administration to patients being in a pre-stage of the condition to be treated in which patients with a pre-form of the corresponding condition is diagnosed.

The term “treatment” is understood the management and care of a patient for the purpose of combating the disease, condition or disorder.

The term “therapeutically effective amount” refers to an amount of a drug or a therapeutic agent that will elicit the desired biological or medical response of a tissue, system or an animal (including man) that is being sought by a researcher or clinician. Non-limiting examples of the desired effect include but are not limited to, at least partially inhibiting or inactivating the AT1 receptor, or the calcium channel blocker, or the rennin inhibitor, or the neutral endopeptidase; or controlling the blood pressure; or lowering the cholesterol level; or treating the cardiovascular or metabolic conditions or diseases, for example, those diseases or conditions described in this application.

The term “synergistic”, as used herein, means that the effect achieved with the methods, combinations and pharmaceutical compositions of the present invention is greater than the sum of the effects that result from individual methods and compositions comprising the active ingredients of this invention separately.

The term “warm-blooded animal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art.

The term “type 2 diabetes” including type 2 diabetes associated with hypertension refers to a disease in which the pancreas does not secrete sufficient insulin due to an impairment of pancreatic beta-cell function and/or in which there is to insensitivity to produced insulin (insulin resistance). Typically, the fasting plasma glucose is less than 126 mg/dL, while pre-diabetes is, e.g., a condition which is characterized by one of following conditions: impaired fasting glucose (110-125 mg/dL) and impaired glucose tolerance (fasting glucose levels less than 126 mg/dL and post-prandial glucose level between 140 mg/dL and 199 mg/dL). Type 2 diabetes mellitus can be associated with or without hypertension. Diabetes mellitus occurs frequently, e.g., in African American, Latino/Hispanic American, Native American, Native American, Asian American and Pacific Islanders. Markers of insulin resistance include HbA1C, HOMA IR, measuring collagen fragments, TGF-β in urine, PAI-1 and prorenin.

The term “hypertension” refers to a condition where the pressure of blood within the blood vessels is higher than normal as it circulates through the body. When the systolic pressure exceeds 150 mmHg or the diastolic pressure exceeds 90 mmHg for a sustained period of time, damage is done to the body. For example, excessive systolic pressure can rupture blood vessels anywhere, and when it occurs within the brain, a stroke results. Hypertension may also cause thickening and narrowing of the blood vessels which ultimately could lead to atherosclerosis.

The term “severe hypertension” refers to hypertension characterized by a systolic blood pressure of ≧180 mmHg and a diastolic blood pressure of ≧110 mmHg.

The term “pulmonary hypertension” (PH) refers to a blood vessel disorder of the lung in which the pressure in the pulmonary artery rises above normal level of ≦25/10 (especially primary and secondary PH), e.g., because the small vessels that supply blood to the lungs constrict or tighten up. According to the WHO, PH may be divided into five categories: pulmonary arterial hypertension (PAH), a PH occurring in the absence of a known cause is referred to as primary pulmonary hypertension, while secondary PH is caused by a condition selected, e.g., from emphysema; bronchitis; collagen vascular diseases, such as scieroderma, Crest syndrome or systemic lupus erythematosus (SLE); PH associated with disorders of the respiratory system; PH due to chronic thrombotic or embolic disease; PH due to disorders directly affecting the pulmonary blood vessels; and pulmonary venous hypertension (PVH).

The term “malignant hypertension” is usually defined as very high blood pressure with swelling of the optic nerve behind the eye, called papilledema (grade IV Keith-Wagner hypertensive retinopathy). This also includes malignant HTN of childhood.

The term “isolated systolic hypertension” refers to hypertension characterized by a systolic blood pressure of ≧140 mmHg and a diastolic blood pressure of <90 mmHg.

The term “familial dyslipidemic hypertension” is characterized by mixed dyslipidemic disorders. Biomarkers include oxidized LDL, HDL, glutathione and homocysteine LPa.

The term “renovascular hypertension” (renal artery stenosis) refers to a condition where the narrowing of the renal artery is significant which leads to an increase of the blood pressure resulting from signals sent out by the kidneys. Biomarkers include renin, PRA and prorenin.

The term “endothelial dysfunction” with or without hypertension refers to a condition in which normal dilation of blood vessels is impaired due to lack of endothelium-derived vasodiiators. Biomarkers include CRP, IL6, ET1, BIG-ET1, VCAM and ICAM. Survival post-MI biomarkers include BNP and procollagen factors.

The term “diastolic dysfunction” refers to abnormal mechanical properties of the heart muscle (myocardium) and includes abnormal left ventricle (LV) diastolic distensibility, impaired filling, and slow or delayed relaxation regardless of whether the ejection fraction is normal or depressed and whether the patient is asymptomatic or symptomatic. Asymptomatic diastolic dysfunction is used to refer to an asymptomatic patient with a normal ejection fraction and an abnormal echo-Doppler pattern of LV filling which is often seen, for example, in patients with hypertensive heart disease. Thus, an asymptomatic patient with hypertensive left ventricular hypertrophy and an echocardiogram showing a normal ejection fraction and abnormal left ventricular filling can be said to have diastolic dysfunction. If such a patient were to exhibit symptoms of effort intolerance and dyspnea, especially if there were evidence of venous congestion and pulmonary edema, it would be more appropriate to use the term diastolic heart failure. This terminology parallels that used in asymptomatic and symptomatic patients with LV systolic dysfunction, and it facilitates the use of a pathophysiologic, diagnostic, and therapeutic framework that includes all patients with LV dysfunction whether or not they have symptoms (William H. Gaasch and Michael R. Zile, Annu. Rev. Med. 55: 373-94, 2004; Gerard P. Aurigemma, William H. Gaasch, N. Engl. J. Med. 351:1097-105, 2004).

The term “cardiac fibrosis” is defined as abnormally high accumulation of collagen and other extracellular matrix proteins due to the enhanced production or decreased degradation of these proteins. Biomarkers include BNP, procollagen factors, LVH, AGE RAGE and CAGE.

The term “peripheral vascular disease” (PVD) refers to the damage or dysfunction of peripheral blood vessels. There are two types of peripheral vascular diseases: peripheral arterial disease (PAD) which refers to diseased peripheral arteries and peripheral venous disorders, which can be measured by an ankle brachial index. PAD is a condition that progressively hardens and narrows arteries due to a gradual buildup of plaque and refers to conditions that effect the blood vessels, such as arteries, veins and capillaries, of the body outside the heart. This is also known as peripheral venous disorder.

The term “atherosclerosis” comes from the Greek words athero (meaning gruel or paste) and sclerosis (hardness). It's the name of the process in which deposits of fatty substances, cholesterol, cellular waste products, calcium and other substances build up in the inner lining of an artery. This buildup is called plaque. It usually affects large and medium-sized arteries. Some hardening of arteries often occurs when people grow older. Plaques can grow large enough to significantly reduce the blood's flow through an artery. But most of the damage occurs when they become fragile and rupture. Plaques that rupture cause blood clots to form that can block blood flow or break off and travel to another part of the body. If either happens and blocks a blood vessel that feeds the heart, it causes a heart attack. If it blocks a blood vessel that feeds the brain, it causes a stroke. And if blood supply to the arms or legs is reduced, it can cause difficulty walking and eventually gangrene.

The term “coronary arterial disease” (CAD) also refers to a condition that progressively hardens and narrows arteries due to a gradual buildup of plaque and refers to conditions that effect the blood vessels such as arteries within the heart. CAD is peculiar form of atherosclerosis that occurs in the three small arteries supplying the heart muscle with oxygen-rich blood. Biomarkers include CPK and Troponin.

The term “cerebrovascular diseases” comprise stroke conditions, such as embolic and thrombotic stroke; large vessel thrombosis and small vessel disease; and hemorrhagic stroke.

The term “embolic stroke” refers to a condition characterized by the formation of blood clots, e.g., in the heart, when clots travel down through the bloodstream in the brain. This may lead to a blockade of small blood vessels and causing a stroke.

The term “thrombotic stroke” refers to a condition where the blood flow is impaired because of a blockade to one or more of the arteries supplying blood to the brain. This process normally leads to thrombosis causing thrombotic strokes. Biomarkers include PAI 1; TPA and platelet function.

The term “metabolic syndrome” (Syndrome X) refers to an overall condition characterized by three or more of the following criteria:
1. abdominal obesity: waist circumference>102 cm in men, and >88 cm in women;
2. hypertriglyceridemia: >150 mg/dL (1.695 mmol/L);
3. low HDL cholesterol: <40 mg/dL (1.036 mmol/L) in men, and <50 mg/dL (1.295 mmol/L) in women;
4. high blood pressure: >130/85 mmHg; and
5. high-fasting glucose: >110 mg/dL (>6.1 mmol/L).

Metabolic syndrome may also be characterized by three or more of the following criteria: triglycerides>150 mg/dL, systolic blood pressure (BP)≧130 mmHg or diastolic BP≧85 mmHg, or on anti-hypertensive treatment, high-density lipoprotein cholesterol<40 mg/dL, fasting blood sugar (FBS)>110 mg/dL, and a body mass index (BMI)>28.8 k/m².

Metabolic syndrome may also be characterized by diabetes, impaired glucose tolerance, impaired fasting glucose, or insulin resistance plus two or more of the following abnormalities:
1. high blood pressure: ≧160/90 mmHg;
2. hyperlipidemia: triglyceride concentration≧150 mg/dL (1.695 mmol/L) and/or HDL cholesterol<35 mg/dL (0.9 mmol/L) in men, and <39 mg/dL (1.0 mmol/L) in women;
3. central obesity: waist-to-hip ratio of >0.90 in men, and >0.85 in women and/or BMI>30 kg/m²; and
4. microalbuminuria: urinary albumin excretion rate≧20 μg/min or an albumin-to-creatinine ratio ≧20 mg/g. Biomarkers include proteinuria, TGF-β, TNF-α and adiponectin.

Biomarkers include LDL, HDL and all the endothelial dysfunction markers.

The term “atrial fibrillation” (AF) refers to a type of irregular or racing heartbeat that may cause blood to collect in the heart and potentially form a clot which may travel to the brain and can cause a stroke.

The term “renal failure”, e.g., chronic renal failure; is characterized, e.g., by proteinuria and/or slight elevation of plasma creatinine concentration (106-177 mmol/L corresponding to 1.2-2.0 mg/dL).

The term “glomerulonephritis” refers to a condition which may be associated with the nephrotic syndrome, a high blood pressure and a decreased renal function, focal, segmental glomerulonephritis, minimal change nephropathy, Lupus nephritis, post-streptococal GN and IgA nephropathy.

The term “nephrotic syndrome” refers to a compilation of conditions including massive proteinuria, edema and central nervous system (CNS) irregularities. Biomarkers include urinary protein excretion.

The term “plaque stabilization” means rendering a plaque less dangerous by preventing, fibrous cap thinning/rupture, smooth muscle cell loss and inflammatory cell accumulation.

The term “renal fibrosis” refers to an abnormal accumulation of collagen and other extracellular matrix proteins, leading to loss of renal function. Biomarkers include collagen fragments and TGF-β in urine.

The term “end-stage renal disease” (ESRD) refers to loss of renal function to the extent that dialysis or renal replacement is needed. Biomarkers include glomerular filtration rate and creatinine clearance.

The term “polycystic kidney disease” (PKD) refers to a genetic disorder characterized by the growth of numerous cysts in the kidney. PKD cysts can slowly reduce much of the mass of kidneys reducing kidney function and leading to kidney failure. PKD may be classified as two major inherited forms of PKD which are autosomal dominant PKD and autosomal recessive PKD, while the non-inherited PKD may be called acquired cystic kidney disease. Biomarkers include reduction of renal cysts by non-invasive imaging.

The term “combination” of an angiotensin II receptor blocker (ARB), or a pharmaceutically acceptable salt thereof; a calcium channel blocker (CCB), or a pharmaceutically acceptable salt thereof; and one of the two active agents selected from a renin inhibitor, or a pharmaceutically acceptable salt thereof; and a neutral endopeptidase (NEP) inhibitor, or a pharmaceutically acceptable salt thereof; means that the components can be administered together as a pharmaceutical composition or as part of the same, unitary dosage form. A combination also includes administering an angiotensin II receptor blocker (ARB), or a pharmaceutically acceptable salt thereof, a calcium channel blocker (CCB), or a pharmaceutically acceptable salt thereof; and one of the two active agents selected from a renin inhibitor, or a pharmaceutically acceptable salt thereof; and a neutral endopeptidase (NEP) inhibitor, or a pharmaceutically acceptable salt thereof; each separately but as part of the same therapeutic regimen. The components, if administered separately, need not necessarily be administered at essentially the same time, although they can if so desired. Thus, a combination also refers, for example, administering an angiotensin II receptor blocker (ARB), or a pharmaceutically acceptable salt thereof; a calcium channel blocker (CCB), or a pharmaceutically acceptable salt thereof; and one of the two active agents selected from a renin inhibitor, or a pharmaceutically acceptable salt thereof; and a neutral endopeptidase (NEP) inhibitor, or a pharmaceutically acceptable salt thereof; as separate dosages or dosage forms, but at the same time. A combination also includes separate administration at different times and in any order.

The renin inhibitors to which the present invention applies are any of those having renin inhibitory activity in vivo and, therefore, pharmaceutical utility, e.g., as therapeutic agents for the prevention of, delay the onset of and/or treatment of hypertension (whether for malignant, essential, reno-vascular, diabetic, isolated systolic, or other secondary type of hypertension), heart failure such as diastolic and congestive heart failure (acute and chronic), left ventricular dysfunction, endothelial dysfunction, diastolic dysfunction, hypertrophic cardiomyopathy, diabetic cardiac myopathy, supraventricular and ventricular arrhythmias, atrial fibrillation (AF), cardiac fibrosis, atrial flutter, detrimental vascular remodeling, plaque stabilization, myocardial infarction (MI) and its sequelae, atherosclerosis including coronary arterial disease (CAD), angina pectoris (whether unstable or stable), renal insufficiency (diabetic and non-diabetic), renal fibrosis, polycystic kidney disease (PKD), type 2 diabetes, metabolic syndrome, secondary aldosteronism, primary and secondary pulmonary hypertension, renal failure conditions such as nephrotic syndrome, diabetic nephropathy, glomerulonephritis, scleroderma, glomerular sclerosis, proteinuria of primary renal disease, renal vascular hypertension, diabetic retinopathy and end-stage renal disease (ESRD), the management of other vascular disorders such as migraine, peripheral vascular disease (PVD), Raynaud's disease, luminal hyperplasia, cognitive dysfunction (such as Alzheimer's), glaucoma and cerebrovascular disease such as embolic or thrombotic stroke.

In particular, the present invention relates to renin inhibitors disclosed in U.S. Pat. No. 5,559,111; No. 6,197,959 and No. 6,376,672, the entire contents of which are incorporated herein by reference.

Suitable angiotensin II receptor blockers which may be employed in the combination of the present invention include AT₁-receptor antagonists having differing structural features, preferred are those with the non-peptidic structures. For example, mention may be made of the compounds that are selected from the group consisting of valsartan (EP 443983), losartan (EP 253310), candesartan (EP 459136), eprosartan (EP 403159), irbesartan (EP 454511), olmesartan (EP 503785), tasosartan (EP 539086), telmisartan (EP 522314), the compound with the designation E-4177 of the formula

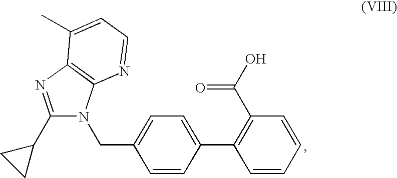

the compound with the designation SC-52458 of the following formula

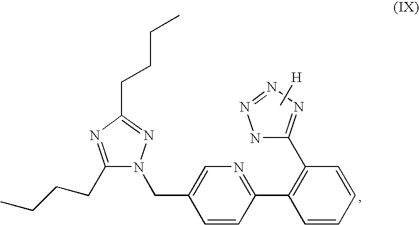

and the compound with the designation the compound ZD-8731 of the formula

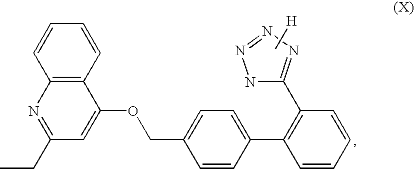

or, in each case, a pharmaceutically acceptable salt thereof.

Preferred AT₁-receptor antagonists are those agents that have reach the market, most preferred is valsartan, or a pharmaceutically acceptable salt thereof.

Suitable Calcium channel which may be employed in the combination of the present invention include the following. The class of CCBs essentially comprises dihydropyridines (DHPs) and non-DHPs such as diltiazem-type and verapamil-type CCBs.

A CCB useful in the combination of the present invention is preferably a DHP representative selected from the group consisting of amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g. as anti-hypertensive, anti-angina pectoris or anti-arrhythmic drugs. Preferred CCBs comprise amlodipine, diltiazem, isradipine, nicardipine, nifedipine, nimodipine, nisoldipine, nitrendipine, and verapamil, or, e.g. dependent on the specific CCB, a pharmaceutically acceptable salt thereof. Especially preferred as DHP is amlodipine or a pharmaceutically acceptable salt, especially the besylate, thereof. An especially preferred representative of non-DHPs is verapamil or a pharmaceutically acceptable salt, especially the hydrochloride, thereof.

The most preferred CCB is amlodipine besylate.

Suitable renin inhibitors useful in the combination of the present invention include compounds having different structural features. For example, mention may be made of compounds which are selected from the group consisting of ditekiren (chemical name: [1S-[1R*,2R*,4R*(1R*,2R*)]]-1-[(1,1-dimethylethoxy)carbonyl]-L-proly 1-L-phenylalanyl-N-[2-hydroxy-5-methyl-1-(2-methylpropyl)-4-[[[2-methyl-1-[[(2-pyridinylmethyl)amino]carbanyl]butyl]amino]carbonyl]hexyl]-N-alfa-methyl-L-histidinamide); terlakiren (chemical name: [R—(R*,S*)]-N-(4-morpholinyloarbonyl)-L-phenylalanyl-N-[1-(cyclohexylmethyl)-2-hydroxy-3-(1-methylethoxy)-3-oxopropyl]-5-methyl-L-cysteineamide); and zankiren (chemical name: [1S-[1R*[R*(R*)],2S*,3R*]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]-amino]-4-thiazolepropanamide), preferably, in each case, the hydrochloride salt thereof.

Preferred renin inhibitor of the present invention include RO 66-1132 and RO 66-1168 of formulae (I) and (II)

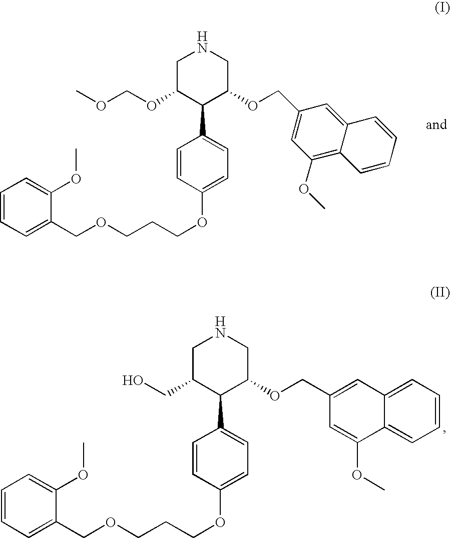

respectively, or a pharmaceutically acceptable salt thereof.

In particular, the present invention relates to a renin inhibitor which is a δ-amino-γ-hydroxy-ω-aryl-alkanoic acid amide derivative of the formula

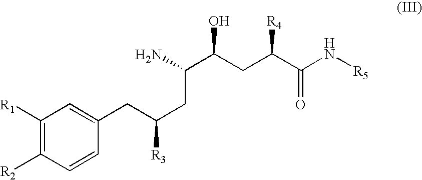

wherein R₁ is halogen, C₁₋₆halogenalkyl, C₁₋₆alkoxy-C₁₋₆alkyloxy or C₁₋₆alkoxy-C₁₋₆-alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

As an alkyl, R₁ may be linear or branched and preferably comprise 1 to 6 C atoms, especially 1 or 4 C atoms. Examples are methyl, ethyl, n- and i-propyl, n-, i- and t-butyl, pentyl and hexyl.

As a halogenalkyl, R₁ may be linear or branched and preferably comprise 1 to 4 C atoms, especially 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

As an alkoxy, R₁ and R₂ may be linear or branched and preferably comprise 1 to 4 C atoms. Examples are methoxy, ethoxy, n- and i-propyloxy, n-, i- and t-butyloxy, pent yloxy and hexyloxy.

As an alkoxyalkyl, R₁ may be linear or branched. The alkoxy group preferably comprises 1 to 4 and especially 1 or 2 C atoms, and the alkyl group preferably comprises 1 to 4 C atoms. Examples are methoxymethyl, 2-methoxyethyl, 3-methoxypropyl, 4-methoxybutyl, 5-methoxypentyl, 6-methoxyhexyl, ethoxymethyl, 2ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

As a C₁₋₆alkoxy-C₁₋₆alkyloxy, R₁ may be linear or branched. The alkoxy group preferably comprises 1 to 4 and especially 1 or 2 C atoms, and the alkyloxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy.

In a preferred embodiment, R₁ is methoxy- or ethoxy-C₁₋₄alkyloxy, and R₂ is preferably methoxy or ethoxy. Particularly preferred are compounds of formula (III), wherein R₁ is 3-methoxypropyloxy and R₂ is methoxy.

As a branched alkyl, R₃ and R₄ preferably comprise 3 to 6 C atoms. Examples are i-propyl, i- and t-butyl, and branched isomers of pentyl and hexyl. In a preferred embodiment, R₃ and R₄ in compounds of formula (III) are in each case i-propyl.

As a cycloalkyl, R₅ may preferably comprise 3 to 8 ring-carbon atoms, 3 or 5 being especially preferred. Some examples are cyclopropyl, cyclobutyl, cyclopentyl, cyclohexyl and cyclooctyl. The cycloalkyl may optionally be substituted by one or more substituents, such as alkyl, halo, oxo, hydroxy, alkoxy, amino, alkylamino, dialkylamino, thiol, alkylthio, nitro, cyano, heterocyclyl and the like.

As an alkyl, R₅ may be linear or branched in the form of alkyl and preferably comprise 1 to 6 C atoms. Examples of alkyl are listed herein above. Methyl, ethyl, n- and i-propyl, n-, i- and t-butyl are preferred.

As a C₁₋₆hydroxyalkyl, R₅ may be linear or branched and preferably comprise 2 to 6 C atoms. Some examples are 2-hydroxyethyl, 2-hydroxypropyl, 3-hydroxypropyl, 2-, 3- or 4-hydroxybutyl, hydroxypentyl and hydroxyhexyl.

As a C₁₋₆alkoxy-C₁₋₆alkyl, R₅ may be linear or branched. The alkoxy group preferably comprises 1 to 4 C atoms and the alkyl group preferably 2 to 4 C atoms. Some examples are 2-methoxyethyl, 2-methoxypropyl, 3-methoxypropyl, 2-, 3- or 4-methoxybutyl, 2-ethoxyethyl, 2-ethoxypropyl, 3-ethoxypropyl, and 2-, 3- or 4-ethoxybutyl.

As a C₁₋₆alkanoyloxy-C₁₋₆alkyl, R₅ may be linear or branched. The alkanoyloxy group preferably comprises 1 to 4 C atoms and the alkyl group preferably 2 to 4 C atoms. Some examples are formyloxymethyl, formyloxyethyl, acetyloxyethyl, propionyloxyethyl and butyroyloxyethyl.

As a C₁₋₆aminoalkyl, R₅ may be linear or branched and preferably comprise 2 to 4 C atoms. Some examples are 2-aminoethyl, 2- or 3-aminopropyl and 2-, 3- or 4-aminobutyl.

As C₁₋₄alkylamino-C₁₋₄alkyl and C₁₋₆dialkylamino-C₁₋₆alkyl, R₅ may be linear or branched. The alkylamino group preferably comprises C₁₋₄alkyl groups and the alkyl group has preferably 2 to 4 C atoms. Some examples are 2-methylaminoethyl, 2-dimethylaminoethyl, 2-ethylaminoethyl, 2-ethylaminoethyl, 3-methylaminopropyl, 3-dimethylaminopropyl, 4-methylaminobutyl and 4-dimethylaminobutyl.

As a HO(O)C—C₁₋₆alkyl, R₅ may be linear or branched and the alkyl group preferably comprises 2 to 4 C atoms. Some examples are carboxymethyl, carboxyethyl, carboxypropyl and carboxybutyl.

As a C₁₋₆alkyl-O—(O)C—C₁₋₆alkyl, R₅ may be linear or branched, and the alkyl groups preferably comprise independently of one another 1 to 4 C atoms. Some examples are methoxycarbonylmethyl, 2-methoxycarbonylethyl, 3-methoxycarbonylpropyl, 4-methoxy-carbonylbutyl, ethoxycafbonylmethyl, 2-ethoxycarbonylethyl, 3-ethoxycarbonylpropyl, and 4-ethoxycarbonylbutyl.

As a H₂N—C(O)—C₁₋₆alkyl, R₅ may be linear or branched, and the alkyl group preferably comprises 2 to 6 C atoms. Some examples are carbamidomethyl, 2-carbamidoethyl, 2-carbamido-2,2-dimethylethyl, 2- or 3-carbamidopropyl, 2-, 3- or 4-carbamidobutyl, 3-carbamido-2-methylpropyl, 3-carbamido-1,2-dimethylpropyl, 3-carbamido-3-ethylpropyl, 3-carbamido-2,2-dimethylpropyl, 2-, 3-, 4- or 5-carbamidopentyl, 4-carbamido-3,3- or -2,2-dimethylbutyl. Preferably, R₅ is 2-carbamido-2,2-dimethylethyl.

Accordingly, preferred are δ-amino-γ-hydroxy-ω-aryl-alkanoic acid amide derivatives of formula (III) having the formula

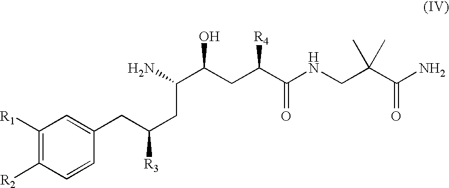

wherein R₁ is 3-methoxypropyloxy; R₂ is methoxy; and R₃ and R₄ are isopropyl; or a pharmaceutically acceptable salt thereof; chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, also known as aliskiren.

The term “aliskiren”, if not defined specifically, is to be understood both as the free base and as a salt thereof, especially a pharmaceutically acceptable salt thereof, most preferably a hemi-fumarate salt thereof.

A suitable NEP inhibitor which may be employed in the combination of the present invention is, e.g., a compound of the formula

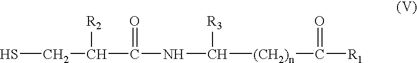

wherein
R₂ is C₁-C₇ alkyl, trifluoromethyl, optionally substituted phenyl or —(CH₂)₁₋₄-(optionally substituted phenyl);
R₃ is hydrogen, C₁-C₇alkyl, optionally substituted phenyl, —(CH₂)₁₋₄-(optionally substituted phenyl);
R₁ is hydroxy, C₁-C₇alkoxy or NH₂;
n is an integer from 1 to 15;
   or pharmaceutically acceptable salt thereof.

The term “optionally substituted phenyl” refers to a phenyl group which may optionally be substituted with s substituent selected from C₁-C₄ alkyl, C₁-C₄ alkoxy, C₁-C₄ alkylthio, hydroxy, Cl, Br, or F.

Preferred selective NEP inhibitors of formula (V) include compounds wherein:
R₂ is benzyl;
R₃ is hydrogen;
n is an integer from 1 to 9;
R₁ is hydroxy;
   or pharmaceutically acceptable salt thereof.

Further preferred is a selective NEP inhibitor of formula (V) which is reported in the literature as SQ 28,603 wherein:
R₂ is benzyl;
R₃ is hydrogen;
n is one; and
R₁ is hydroxy.

The preparation of selective NEP inhibitors of formula (V) wherein R₂ is other than trifluoromethyl is disclosed by Delaney et al. in U.S. Pat. No. 4,722,810. The preparation of selective NEP inhibitors of formula (VI) wherein R₂ is trifluoromethyl is disclosed by Delaney et al. in U.S. Pat. No. 5,223,516.

Further NEP inhibitors within the scope of the present invention include compounds disclosed in U.S. Pat. No. 4,610,816, herein incorporated by reference, including in particular N—[N-[1(S)-carboxyl-3-phenylproplyl]-(S)-phenylalanyl]-(S)-isoserine and N—[N-[((1S)-carboxy-2-phenyl)ethyl]-(S)-phenylalanyl]-β-alanine; compounds disclosed in U.S. Pat. No. 4,929,641, in particular N-[2(S)-mercaptomethyl-3-(2-methylphenyl)-propionyl]-methionine; SQ 28,603 (N-[2-(mercaptomethyl)-1-oxo-3-phenylpropyl]-β-alanine), disclosed in South African Patent Application 84/0670; UK 69578 (cis-4-[[[1-[2-carboxy-3-(2-methoxyethoxy)-propyl]-cyclopentyl]carbonyl]amino]-cyclohexanecarboxylic acid) and its active enantiomer(s); thiorphan and its enantiomers; retro-thiorphan; phosphoramidon; and SQ 29,072 (7-[[2-(mercaptomethyl)-1-oxo-3-phenylpropyl]amino]-heptanoic acid). Also suitable for use are any pro-drug forms of the above-listed NEP inhibitors, e.g., compounds in which one or more carboxylic acid groups are esterified.

NEP inhibitors within the scope of the present invention also include the compounds disclosed in U.S. Pat. No. 5,217,996, particularly, N-(3-carboxy-1-oxopropyl)-(4S)-p-phenylphenylmethyl)-4-amino-(2R)-methylbutanoic acid ethyl ester and N-(3-carboxy-1-oxopropyl)-(4S)-p-phenylphenylmethyl)-4-amino-(2R)-methylbutanoic acid, or in each case, a pharmaceutically acceptable salt thereof; the compounds disclosed in EP 00342850, particularly (S)-cis-4-[1-[2-(5-indanyloxycarbonyl)-3-(2-methoxyethoxy)propyl]-1-cyclopentanecarboxamido]-1-cyclohexanecarboxylic acid; the compounds disclosed in GB 02218983, particularly 3-(1-[6-endo-hydroxymethylbicyclo[2,2,1]heptane-2-exo-carbamoyl]-cyclopentyl)-2-(2-methoxyethyl)propanoic acid; the compounds disclosed in WO 92/14706, particularly M-(1-(3-(N-t-butoxycarbonyl-(S)-prolylamino)-2(S)-t-butoxy-carbonylpropyl)-cyclopentanecarbonyl)-O-benzyl-(S)-serine methyl ester; the compounds disclosed in EP 00343911; the compounds disclosed in JP 06234754; the compounds disclosed in EP 00361365, particularly 4-[[2-(mercaptomethyl)-1-oxo-3-phenylpropyl]amino]benzoic acid; the compounds disclosed in WO 90/09374, particularly 3-[1-(cis-4-carboxycarbonyl-cis-3-butylcyclohexyl-r-1-carboamoyl)cyclopentyl]-2S-(2-methoxyethoxymethyl)propanoic acid; the compounds disclosed in JP 07157459, particularly N-((2S)-2-(4-biphenylmethyl)-4-carboxy-5-phenoxyvaleryl)glycine; the compounds disclosed in WO 94/15908 particularly N-(1N-hydroxycarbamoylmethyl)-1-cyclopentanecarbonyl)-L-phenylalanine; the compounds disclosed in U.S. Pat. No. 5,273,990 particularly (S)-(2-biphenyl-4-yl)-1-(1H-tetrazol-5-yl)ethylamino) methylphosphonic acid; the compounds disclosed in U.S. Pat. No. 5,294,632 particularly (S)-5-(N-(2-(phosphonomethylamino)-3-(4-biphenyl)propionyl)-2-aminoethyl)tetrazole; the compounds disclosed in U.S. Pat. No. 5,250,522, particularly β-alanine, 3-[1,1′-biphenyl]-4-yl-N-[diphenoxyphosphinyl)methyl]-L-alanyl; the compounds disclosed in EP 00636621, particularly N-(2-carboxy-4-thienyl)-3-mercapto-2-benzylpropanamide; the compounds disclosed in WO 93/09101, particularly 2-(2-mercaptomethyl-3-phenylpropionamido)thiazol-4-ylcarboxylic acid; the compounds disclosed in EP 00590442 particularly ((L)-(1-((2,2-dimethyl-1,3-dioxolan-4-yl)-methoxy)carbonyl)-2-phenylethyl)-L-phenylalanyl)-β-alanine, N—[N-[(L)-[1-[(2,2-dimethyl-1,3-dioxolan-4-yl)-methoxy]carbonyl]-2-phenylethyl]-L-phenylalanyl]-(R)-alanine, N—[N-[(L)-1-carboxy-2-phenylethyl]-L-phenylalanyl]-(R)-alanine, N-[2-acetylthiomethyl-3-(2-methyl-phenyl)-propionyl]-methionine ethyl ester, N-[2-mercaptomethyl-3-(2-methylphenyl)propioyl]-methionine, N-[2(S)-mercaptomethyl-3-(2-methylphenyl)propanoyl]-(S)-isoserine, N—(S)-[3-mercapto-2-(2-methylphenyl)propionyl]-(S)-2-methoxy-(R)-alanine, N-[1-[[1(S)-benzyl oxycarbonyl-3-phenylpropyl]amino]cyclopentylcarbonyl]-(S)-isoserine, N-[1-[[1(S)-carbonyl-3-phenylpropy]amino]-cyclopentylcarbonyl]-(S)-isoserine, 1,1′-[dithiobis-[2(S)-(2-methylbenzyl)-1-oxo-3,1-propanediyl]]-bis-(S)-isoserine, 1,1′-[dithiobis-[2(S)-(2-methylbenzyl)-1-oxo-3,1-propanediyl]]-bis-(S)-methionine, N-(3-phenyl-2-(mercaptomethyl)-propionyl)-(S)-4-(methylmercapto)methionine, N-[2-acetylthiomethyl-3-phenyl-propionyl]-3-aminobenzoic acid, N-[2-mercaptomethyl-3-phenyl-propionyl]-3-aminobenzoic acid, N-[1-(2-carboxy-4-phenylbutyl)-cyclopentanecarbonyl]-(S)-isoserine, N-[1-(acetylthiomethyl)-cyclopentane-carbonyl]-(S)-methionine ethyl ester, 3(S)-[2-(acetylthiomethyl)-3-phenyl-propionyl]amino-ε-caprolactam; and the compounds disclosed in WO 93/10773, particularly, N-(2-acetylthiomethyl-3-(2-methylphenyl)propionyl)-methionine ethyl ester.

Especially suitable NEP inhibitors include N-(3-carboxy-1-oxopropyl)-(4S)-p-phenylphenylmethyl)-4-amino-(2R)-methylbutanoic acid ethyl ester and N-(3-carboxy-1-oxopropyl)-(4S)-p-phenylphenylmethyl)-4-amino-(2R)-methylbutanoic acid of the formulae.

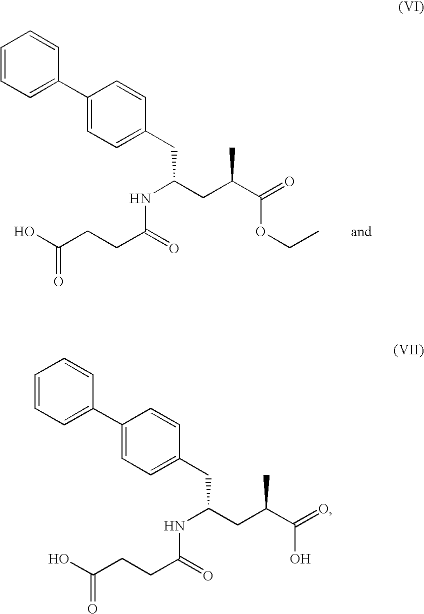

respectively, or in each case, a pharmaceutically acceptable salt thereof. Preferred salts of the compound of formula (VI) include, put are not limited to, a sodium salt disclosed in U.S. Pat. No. 5,217,996; and a triethanolamine or a tris(hydroxymethyl)aminomethane salt disclosed in WO 03/059345.

The subject matter relating to NEP inhibitors referred herein above, e.g., in U.S. patents and EP, GB, JP or WO patent applications, is herewith incorporated by reference, especially the subject matter corresponding to NEP inhibitors, and pharmaceutically acceptable salts and pharmaceutical compositions thereof, that are disclosed herein.

The combination of the present invention may comprise in addition a diuretic. A diuretic is, for example, a thiazide derivative selected from the group consisting of chlorothiazide, hydrochlorothiazide, methylclothiazide, and chlorothalidon. The most preferred diuretic is hydrochlorothiazide. A diuretic furthermore is a potassium sparing diuretic such as amiloride or triameterine, or a pharmaceutically acceptable salt thereof.

Preferred is a combination according to the present invention comprising an angiotensin II blocker, e.g., valsartan, or a pharmaceutically acceptable salt thereof; a calcium channel blocker, e.g., amlodipine, especially in the form of the besylate salt thereof; and a renin inhibitor, e.g., aliskiren, especially in the form of the hemi-fumarate salt thereof.

Preferred is also a combination according to the present invention comprising an angiotensin II blocker, e.g., valsartan, or a pharmaceutically acceptable salt thereof; a calcium channel blocker, e.g., amlodipine, especially in the form of the besylate salt thereof; and a NEP inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid, or a pharmaceutically acceptable salt thereof.

Preferred is also a combination according to the present invention comprising an angiotensin II blocker, e.g., valsartan, or a pharmaceutically acceptable salt thereof; a calcium channel blocker, e.g., amlodipine, especially in the form of the besylate salt thereof; a renin inhibitor, e.g., aliskiren, especially in the form of the hemi-fumarate salt thereof; and a diuretic, e.g., hydrochlorothiazide.

Preferred is also a combination according to the present invention comprising an angiotensin II blocker, e.g., valsartan, or a pharmaceutically acceptable salt thereof; a calcium channel blocker, e.g., amlodipine, especially in the form of the besylate salt thereof; a NEP inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid, or a pharmaceutically acceptable salt thereof; and a diuretic, e.g., hydrochlorothiazide.

As indicated herein above, the compounds to be combined may be present as their pharmaceutically acceptable salts. If these compounds have, e.g., at least one basic center such as an amino group, they can form acid addition salts thereof. Similarly, the compounds having at least one acid group (for example COOH) can form salts with bases. Corresponding internal salts may furthermore be formed, if a compound comprises, e.g., both a carboxy and an amino group.

The corresponding active ingredients or a pharmaceutically acceptable salts may also be used in form of a solvate, such as a hydrate or including other solvents used, e.g., in their crystallization.

Furthermore, the present invention provides pharmaceutical compositions comprising:
(a) an angiotensin II receptor blocker (ARB), or a pharmaceutically acceptable salt thereof;
(b) a calcium channel blocker (CCB), or a pharmaceutically acceptable salt thereof; and
(c) one of the two active agents selected from
   (i) a renin inhibitor, or a pharmaceutically acceptable salt thereof; and
   (ii) a neutral endopeptidase (NEP) inhibitor, or a pharmaceutically acceptable salt thereof
      and a pharmaceutically acceptable carrier.

As disclosed herein above, an angiotensin II receptor blocker (ARB), e.g., valsartan, or a pharmaceutically acceptable salt thereof; a calcium channel blocker (CCB), e.g., amlodipine, preferably in the form of the besylate salt thereof; and one of the two active agents selected from a renin inhibitor, in particular, aliskiren, preferably in the form of the hemi-fumarate salt thereof; and a neutral endopeptidase (NEP) inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid, or a pharmaceutically acceptable salt thereof; and optionally a diuretic, e.g., hydrochlorothiazide, may be co-administered as a pharmaceutical composition. The components may be administered together in any conventional dosage form, usually also together with a pharmaceutically acceptable carrier or diluent.

The pharmaceutical compositions according to the invention are those suitable for enteral, such as oral or rectal, transdermal and parenteral administration to mammals, including man. For oral administration the pharmaceutical composition comprising an angiotensin II receptor blocker (ARB), e.g., valsartan, or a pharmaceutically acceptable salt thereof; a calcium channel blocker (CCB), e.g., amlodipine, preferably in the form of the besylate salt thereof; and one of the two active agents selected from a renin inhibitor, in particular, aliskiren, preferably in the form of the hemi-fumarate salt thereof; and a neutral endopeptidase (NEP) inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methyl butanoic acid, or a pharmaceutically acceptable salt thereof; and optionally a diuretic, e.g., hydrochlorothiazide, can take the form of solutions, suspensions, tablets, pills, capsules, powders, microemulsions, unit dose packets and the like. Preferred are tablets and gelatin capsules comprising the active ingredient together with: a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-90%, preferably about 1-80%, of the active ingredient.

The dosage of the active ingredients can depend on a variety of factors, such as mode of administration, homeothermic species, age and/or individual condition.

Preferred dosages for the active ingredients of the pharmaceutical combinations according to the present invention are therapeutically effective dosages, especially those which are commercially available.

Normally, in the case of oral administration, an approximate daily dose of from about 1 mg to about 360 mg is to be estimated, e.g., for a patient of approximately 75 kg in weight.

For example, angiotensin II receptor blockers, e.g., valsartan, are supplied in the form of a suitable dosage unit form, e.g., a capsule or tablet, and comprising a therapeutically effective amount of an angiotensin II receptor blocker, e.g., from about 20 to about 320 mg, of valsartan, which may be applied to patients. The application of the active ingredient may occur up to three times a day, starting, e.g., with a daily dose of 20 mg or 40 mg of an angiotensin II receptor blocker, e.g., valsartan, increasing via 80 mg daily and further to 160 mg daily, and finally up to 320 mg daily. Preferably, an angiotensin II receptor blocker, e.g., valsartan is applied once a day or twice a day with a dose of preferably 80 mg or 160 mg, respectively, each. Corresponding doses may be taken, e.g., in the morning, at mid-day or in the evening.

In the case of calcium channel blockers, preferred dosage unit forms are, e.g., tablets or capsules, comprising about 1.0 mg to about 180 mg, preferably about 2.5 mg to about 50 mg, more preferably 2.5 to 10 mg, of the CCB, e.g. amlodipine, depending on the specific CCB. Preferred dosage unit forms are, for example, tablets or capsules comprising e.g. from about 1 mg to about 40 mg, preferably 2.5 to 20 mg, more preferably 2.5 to 10 mg, daily especially when the CCB, in particular amlodipine, is administered orally.

The doses of renin inhibitors, e.g. aliskiren, to be administered to warm-blooded animals, including man, of approximately 75 kg body weight, especially the doses effective for the inhibition of renin activity, e.g., in lowering blood pressure, are from preferably about 3 mg to about 3 g, more preferably from about 10 mg to about 1 g, e.g., from 20 to 200 mg/person/day, divided preferably into 1 to 4 single doses which may, e.g., be of the same size. Usually, children receive about half of the adult dose. The dose necessary for each individual can be monitored, e.g., by measuring the serum concentration of the active ingredient, and adjusted to an optimum level. Single doses comprise, e.g., 75 mg, 150 mg or 300 mg per adult patient.

In the case of NEP inhibitors, preferred dosage unit forms are, e.g., tablets or capsules comprising, e.g., from about 20 mg to about 800 mg, preferably from about 50 mg to about 700 mg, even more preferably from about 100 mg to about 600 mg, and most preferably from about 100 mg to about 300 mg, of the NEP inhibitor administered preferably once a day.

In case of diuretics, preferred dosage unit forms are, e.g., tablets or capsules comprising, e.g., from about 5 mg to about 50 mg, preferably from about 6.25 mg to about 25 mg. A daily dose of 6.25 mg, 12.5 mg or 25 mg of e.g. hydrochlorothiazide is preferably administered once a day.

The above doses encompass a therapeutically effective amount of the active ingredients of the present invention

An example of a preferred composition, comprises an amount of Valsartan between 60 and 100 mg e.g. 80 mg, an amount of amlodipine between 2 and 12 mg e.g. 2.5 or 5 mg, an amount of aliskiskiren of 20 to 200 mg, e.g., 75 mg, 150 mg or 300 mg, and an amount of HCTZ between 8 and 16 mg e.g. 12.5 mg.

Another example of a preferred composition, comprises an amount of Valsartan between 140 and 180 mg e.g. 160 mg, an amount of amlodipine between 2 and 12 mg e.g. 2.5 or 5 or 10 mg, amount of aliskiskiren of 20 to 200 mg, e.g., 75 mg, 150 mg or 300 mg, and an amount of HCTZ between 8 and 16 mg e.g. 12.5 mg.

Another example of a preferred composition comprises an amount of Valsartan between 140 and 180 mg e.g. 160 mg, an amount of amlodipine between 4 and 12 mg e.g. 5 mg or 10 mg, amount of aliskiskiren of 20 to 200 mg, e.g., 75 mg, 150 mg or 300 mg, and an amount of HCTZ between 20 and 30 mg e.g. 25 mg.

Since the present invention has an aspect that relates to methods for the prevention of, delay the onset of and/or treatment with a combination of compounds which may be administered separately, the invention also relates to combining separate pharmaceutical compositions in a kit form. The kit may comprise, e.g., three to four separate pharmaceutical compositions: (1) a composition comprising an angiotensin II blocker, or a pharmaceutically acceptable salt thereof, and a pharmaceutically acceptable carrier or diluent; (2) a composition comprising a CCB, or a pharmaceutically acceptable salt thereof, and a pharmaceutically acceptable carrier or diluent; (3) a composition comprising one of the two active agents selected from a renin inhibitor, or a pharmaceutically acceptable salt thereof, and a NEP inhibitor, or a pharmaceutically acceptable salt thereof; and a pharmaceutically acceptable carrier or diluent; and (4) optionally a diuretic, or a pharmaceutically acceptable salt thereof, and a pharmaceutically acceptable carrier or diluent. The amounts of (1), (2), (3) and (4) are such that, when co-administered separately a beneficial therapeutic effect(s) is achieved. The kit comprises a container for containing the separate compositions such as a divided bottle or a divided foil packet, wherein each compartment contains a plurality of dosage forms (e.g., tablets) comprising, e.g., (1), (2) or (3). Alternatively, rather than separating the active ingredient-containing dosage forms, the kit may contain separate compartments each of which contains a whole dosage which in turn comprises separate dosage forms. An example of this type of kit is a blister pack wherein each individual blister contains three or four (or more) tablets, one (or more) tablet(s) comprising a pharmaceutical composition (1), the second (or more) tablet(s) comprising a pharmaceutical composition (2), the third (or more) tablet(s) comprising a pharmaceutical composition (3) and optionally the forth (or more) tablet(s) comprising a pharmaceutical composition (4). Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. In the case of the instant invention a kit may, e.g., comprise:

(1) a therapeutically effective amount of a composition comprising an angiotensin II blocker, e.g., valsartan, or a pharmaceutically acceptable salt thereof, and a pharmaceutically acceptable carrier or diluent, in a first dosage form;
(2) a therapeutically effective amount of a composition comprising a CCB, in particular, amlodipine, preferably in the form of the besylate salt thereof, and a pharmaceutically acceptable carrier or diluent, in a second dosage form;
(3) a therapeutically effective amount of a a composition comprising one of the two active agents selected from a renin inhibitor, in particular, aliskiren, preferably in the form of the hemi-fumarate salt thereof, and a pharmaceutically acceptable carrier or diluent, and a neutral endopeptidase (NEP) inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid; or a pharmaceutically acceptable salt thereof; in an amount such that, following administration, a beneficial therapeutic effect(s) is achieved, and a pharmaceutically acceptable carrier or diluent, in a third dosage form;
(4) optionally a diuretic, e.g., hydrochlorothiazide, or a pharmaceutically acceptable salt thereof, in a forth dosage form; and
(5) a container for containing said first, second, third and optionally forth dosage forms.

The present invention further relates to a method for the prevention of, delay the onset of and/or treatment of a disease or a condition mediated by angiotensin II and/or to NEP activity, which method comprises administering to a warm-blooded animal, including man, in need thereof, a therapeutically effective amount of a pharmaceutical composition comprising:
(a) an angiotensin II receptor blocker (ARB), or a pharmaceutically acceptable salt thereof;
(b) a calcium channel blocker (CCB), or a pharmaceutically acceptable salt thereof; and
(c) one of the two active agents selected from
   (i) a renin inhibitor, or a pharmaceutically acceptable salt thereof; and
   (ii) a neutral endopeptidase (NEP) inhibitor, or a pharmaceutically acceptable salt thereof;
   and a pharmaceutically acceptable carrier.

It has surprisingly been found that, a combination of an angiotensin II receptor blocker (ARB), e.g., valsartan, or a pharmaceutically acceptable salt thereof; a calcium channel blocker (CCB), e.g., amlodipine, preferably in the form of the besylate salt thereof; and one of the two active agents selected from a renin inhibitor, in particular, aliskiren, preferably in the form of the hemi-fumarate salt thereof; and a neutral endopeptidase (NEP) inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid, or a pharmaceutically acceptable salt thereof; and optionally a diuretic, e.g., hydrochlorothiazide, achieves greater therapeutic effect than the administration of an angiotensin II blocker, a CCB, a renin inhibitor, a NEP inhibitor or a diuretic alone. Greater efficacy can also be documented as a prolonged duration of action. The duration of action can be monitored as either the time to return to baseline prior to the next dose or as the area under the curve (AUC) and is expressed as the product of the change in blood pressure in millimeters of mercury (change in mmHg) and the duration of the effect (min, hours or days).

Further benefits are that lower doses of the individual drugs to be combined according to the present invention can be used to reduce the dosage, e.g., that the dosages need not only often be smaller but are also applied less frequently, or can be used to diminish the incidence of side effects. The combined administration of an angiotensin II receptor blocker (ARB), e.g., valsartan, or a pharmaceutically acceptable salt thereof; a calcium channel blocker (CCB), e.g., amlodipine, preferably in the form of the besylate salt thereof; and one of the two active agents selected from a renin inhibitor, in particular, aliskiren, preferably in the form of the hemi-fumarate salt thereof; and a neutral endopeptidase (NEP) inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid, or a pharmaceutically acceptable salt thereof; and optionally a diuretic, e.g., hydrochlorothiazide, results in a significant response in a greater percentage of treated patients, i.e., a greater responder rate results, regardless of the underlying etiology of the condition. This is in accordance with the desires and requirements of the patients to be treated.

It can be shown that combination therapy with an angiotensin II receptor blocker (ARB), e.g., valsartan, or a pharmaceutically acceptable salt thereof; a calcium channel blocker (CCB), e.g., amlodipine, preferably in the form of the besylate salt thereof; and one of the two active agents selected from a renin inhibitor, in particular, aliskiren, preferably in the form of the hemi-fumarate salt thereof; and a neutral endopeptidase (NEP) inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid, or a pharmaceutically acceptable salt thereof; and optionally a diuretic, e.g., hydrochlorothiazide, results in a more effective antihypertensive therapy (whether for malignant, essential, reno-vascular, diabetic, isolated systolic, or other secondary type of hypertension) through improved efficacy as well as a greater responder rate. The combination is also useful in the prevention of, delay the onset of and/or treatment of heart failure such as (acute and chronic) congestive heart failure, left ventricular dysfunction, diastolic dysfunction, hypertrophic cardiomyopathy, diabetic cardiac myopathy, supraventricular and ventricular arrhythmias, atrial fibrillation (AF), atrial flutter, detrimental vascular remodeling or plaque stabilization. It can further be shown that a therapy with an angiotensin II receptor blocker (ARB) and a calcium channel blocker (CCB), and one of the two active agents selected from a renin inhibitor and a neutral endopeptidase (NEP) inhibitor and optionally a diuretic proves to be beneficial in the treatment and prevention of myocardial infarction and its sequelae. A combination of the present invention is also useful in treating atherosclerosis including coronary arterial disease (CAD), angina pectoris (whether unstable or stable), renal insufficiency (diabetic and non-diabetic), renal fibrosis, polycystic kidney disease (PKD) and metabolic syndrome. In addition, combination therapy using a combination of the present invention can improve endothelial dysfunction, thereby providing benefit in diseases in which normal endothelial function is disrupted such as heart failure, angina pectoris and type 2 diabetes. Furthermore, a combination of the present invention may be used for the prevention of, delay the onset of and/or treatment of secondary aldosteronism, primary and secondary pulmonary hypertension, renal failure conditions such as nephrotic syndrome, diabetic nephropathy, glomerulonephritis, scleroderma, glomerular sclerosis, proteinuria of primary renal disease, renal vascular hypertension, diabetic retinopathy and end-stage renal disease (ESRD), the management of other vascular disorders such as migraine, peripheral vascular disease (PVD), Raynaud's disease, luminal hyperplasia, cognitive dysfunction (such as Alzheimer's), glaucoma and cerebrovascular disease such as embolic or thrombotic stroke.

The structure of the active agents identified by generic or tradenames or code numbers may be taken from the actual edition of the standard compendium “The Merck Index” or from databases, e.g., Life Cycle Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The invention furthermore relates to the use of a combination according to the present invention for the manufacture of a medicament for the prevention of, delay the onset of and/or treatment of cardiovascular disorders.

Accordingly, another embodiment of the present invention relates to the use of a combination according to the invention for the manufacture of a medicament for the prevention of, delay the onset of and/or treatment of cardiovascular disorders, especially a disease or a condition selected from the group consisting of hypertension (whether for malignant, essential, reno-vascular, diabetic, isolated systolic, or other secondary type of hypertension), heart failure such as diastolic and congestive heart failure (acute and chronic), left ventricular or endothelial dysfunction, hypertrophic cardiomyopathy, diabetic cardiac myopathy, supraventricular and ventricular arrhythmias, atrial fibrillation (AF), cardiac fibrosis, atrial flutter, detrimental vascular remodeling, plaque stabilization, myocardial infarction (MI) and its sequelae, atherosclerosis including coronary arterial disease (CAD), angina pectoris (whether unstable or stable), renal insufficiency (diabetic and non-diabetic), renal fibrosis, polycystic kidney disease (PKD), type 2 diabetes, metabolic syndrome, secondary aldosteronism, primary and secondary pulmonary hypertension, renal failure conditions such as nephrotic syndrome, diabetic nephropathy, glomerulonephritis, scleroderma, glomerular sclerosis, proteinuria of primary renal disease, renal vascular hypertension, diabetic retinopathy and end-stage renal disease (ESRD), the management of other vascular disorders such as migraine, peripheral vascular disease (PVD), Raynaud's disease, luminal hyperplasia, cognitive dysfunction (such as Alzheimer's), glaucoma and cerebrovascular disease such as embolic or thrombotic stroke.

Especially, a combination according to the present invention may be employed, e.g., for the prevention of, delay the onset of and/or treatment of diseases and conditions selected from the group as specified above, and also diseases, illnesses, conditions or symptoms related to, or encountered or associated therewith.

Preferably, a combination according to the present invention may be employed for the treatment of hypertension, congestive heart failure, atherosclerosis, endothelial dysfunction, and renal insufficiency.

In particular, all the more surprising is the experimental finding that a combination of the present invention results in a beneficial, especially a synergistic, therapeutic effect but also in benefits resulting from combined treatment such as a surprising prolongation of efficacy, a broader variety of therapeutic treatment and surprising beneficial effects on diseases and conditions as specified hereinbefore or hereinafter.

The pharmaceutical activities as effected by administration of a an angiotensin II receptor blocker (ARB), e.g., valsartan, or a pharmaceutically acceptable salt thereof; a calcium channel blocker (CCB), e.g., amlodipine, preferably in the form of the besylate salt thereof; and one of the two active agents selected from a renin inhibitor, in particular, aliskiren, preferably in the form of the hemi-fumarate salt thereof; and a neutral endopeptidase (NEP) inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid, or a pharmaceutically acceptable salt thereof; and optionally a diuretic, e.g., hydrochlorothiazide, respectively, or by administration of a combination of therapeutic agents used according to the present invention may be demonstrated, e.g., by using corresponding pharmacological models well-known in the pertinent art. A person skilled in the art is fully enabled to select a relevant test model to prove the hereinbefore and hereinafter indicated therapeutic indications and beneficial effects.

A combination according to the present invention can be administered by various routes of administration. Each agent can be tested over a wide-range of dosages to determine the optimal drug level for each therapeutic agent in the specific combination to elicit the maximal response. For these studies, it is preferred to use treatment groups consisting of at least 6 animals per group. Each study is best performed in away wherein the effects of the combination treatment group are determined at the same time as the individual components are evaluated. Although drug effects may be observed with acute administration, it is preferable to observe responses in a chronic setting. The long-term study is of sufficient duration to allow for the full development of compensatory responses to occur and, therefore, the observed effect will most likely depict the actual responses of the test system representing sustained or persistent effects.

Representative studies may be carried out with a combination of an angiotensin II receptor blocker (ARB), e.g., valsartan, or a pharmaceutically acceptable salt thereof; a calcium channel blocker (CCB), e.g., amlodipine, preferably in the form of the besylate salt thereof; and one of the two active agents selected from a renin inhibitor, in particular, aliskiren, preferably in the form of the hemi-fumarate salt thereof; and a neutral endopeptidase (NEP) inhibitor, e.g., N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid, or a pharmaceutically acceptable salt thereof; and optionally a diuretic, e.g., hydrochlorothiazide, e.g., applying the following methodology:

Drug efficacy is assessed in various animal models including the deoxycorticosterone acetate-salt rat (DOCA-salt), the Dahl salt-sensitive (DS) and salt-resistant (DR) rat, and the spontaneously hypertensive rat (SHR), either maintained on a normal salt diet or with salt loading (4-8% salt in rat chow or 1% NaCl as drinking water).

The DOCA-salt test model utilizes either an acute or chronic study protocol. An acute study procedure involves assessment of the effects of various test substances over a six-hour experimental period using rats with indwelling femoral arterial and venous catheters. The Acute Study Procedure evaluates test substances for their ability to reduce blood pressure during the established phase of DOCA-salt hypertension. In contrast, the Chronic Study Procedure assesses the ability of test substances to prevent or delay the rise in blood pressure during the development phase of DOCA-salt hypertension. Therefore, blood pressure will be monitored in the chronic study procedure by means of a radiotransmitter. The radiotransmitter is surgically implanted into the abdominal aorta of rats, prior to the initiation of DOCA-salt treatment and thus, prior to the induction of hypertension. Blood pressure is chronically monitored for periods of up 6 weeks (approximately one week prior to DOCA-salt administration and for 5 weeks thereafter).

Rats are anesthetized with 2-3% isoflurane in oxygen inhalant followed by Amytal sodium (amobarbital) 100 mg/kg, ip. The level of anesthesia is assessed by a steady rhythmic breathing pattern.

### Acute Study Procedure:

Rats undergo a unilateral nephrectomy at the time of DOCA implantation. Hair is clipped on the left flank and the back of the neck and scrubbed with sterile alcohol swabs and povidone/iodine. During surgery rats are placed on a heating pad to maintain body temperature at 37° C.

A 20 mm incision is made through the skin and underlying muscle to expose the left kidney. The kidney is freed of surrounding tissue, exteriorized and two ligatures (3-0 silk) are tied securely around the renal artery and vein proximal to their juncture with the aorta. The renal artery and vein are then severed and the kidney removed. The muscle and skin wounds are closed with 4-0 silk suture and stainless steel wound clips, respectively. At the same time, a 15 mm incision is made on the back of the neck and a 3-week-release pellet (Innovative Research of America, Sarasota, Fla.) containing deoxycorticosterone acetate (100 mg/kg) is implanted subcutaneously. The wound is then closed with stainless-steel clips and both wounds are treated with povidone/iodine; the rats are given a post-surgical intramuscular injection of procaine penicillin G (100,000 U) and buprenorphine (0.05-0.1 mg/kg) s.c. The rats are immediately placed on 1% NaCl+0.2% KCl drinking water; this treatment continues for at least 3 weeks at which time the animals have become hypertensive and available for experimentation.

Forty-eight hours prior to experimentation, animals are anesthetized with isoflurane and catheters are implanted in the femoral artery and vein for measuring arterial pressure, collection of blood, and administration of test compounds. Rats are allowed to recover for 48 hours while tethered in a Plexiglas home cage, which also serves as the experimental chamber.

### Chronic Study Procedure:

This procedure is the same as above except that rats are implanted with a radiotransmitter, 7-10 days prior to the unilateral nephrectomy and initiation of DOCA and salt. In addition, rats do not undergo surgery for placement of femoral arterial and venous catheters. Radiotransmitters are implanted as described by M. K. Bazil, C. Krulan and R. L. Webb. in J. Cardiovasc. Pharmacol. 22: 897-905, 1993.

Protocols are then set-up on the computer for measurement of blood pressure, heart rate, etc, at predetermined time points. Baseline data is collected at various time points and over various time intervals. For example, baseline or pre-dose values usually consist of data collection and averaging over 3 consecutive, 24-hour time periods prior to drug administration.

Blood pressure, heart rate and activity are determined at various pre-selected time points before, during, and after drug administration. All measurements are performed in unrestrained and undisturbed animals. The maximum study time, determined by battery life, could be as long as nine months. For studies of this duration, rats are dosed orally (1-3 ml/kg vehicle), no more than twice daily or drug is administered via the drinking water or mixed with food. For studies of a shorter duration, that is, up to 8 weeks, drugs are given via subcutaneously implanted osmotic minipumps. Osmotic minipumps are selected based on drug delivery rate and time.

The Dahl salt-sensitive (DSS) and salt-resistant (DSR) rat may also be utilized for the study of the combinations according to the present invention. The DSR rat is commonly used as a normotensive control for these studies. For the study of various combinations, a typical protocol is defined as follows:

Dahl salt-sensitive (DSS) rats are 6 weeks of age upon arrival to our animal facilities. Radiotransmitters are implanted into Dahl salt-sensitive rats at 7 weeks of age. All animals are placed on a high salt diet (8%) between 7 and 8 weeks of age. Drug treatment is initiated at 9 weeks of age and is continued for 3 weeks. Drugs are administered once daily by oral gavage but may also be given by other routes (e.g., intra-peritoneal, intra-venous, or subcutaneous). Dahl salt-sensitive rats are randomized to receive one of the various treatments specified above. Drugs are administered by oral gavage, once daily in the morning for 3 weeks. Blood pressure (mean, systolic, and diastolic) and heart rate are continuously monitored, 24 hours per day for the full duration of the study using radiotelemetric procedures. All values depict 24 hour average responses for each animal but data summarization may also be performed using other time intervals, for example, hourly averaging. Body weights are recorded at weekly intervals. Upon completion of the study, all rats are sacrificed and hearts are removed, sectioned and weighed. Cardiac mass is determined as the left ventricular weight to body weight ratio for each animal within a treatment group. Other tissues, including but not restricted to the kidney, may be removed at sacrifice for determination of biochemical markers, to assess the extent of tissue damage (histology, immunohistochemistry, etc), and for gene expression profiling.

Additionally, SHR are utilized to study the effects of the claimed combination. The hypertensive background of the SHR is modified either by chronic salt loading in an effort to suppress the renin angiotensin system (RAS) or chronic salt depletion to activate the RAS in the SHR. These manipulations will be carried out to more extensively evaluate the efficacy of the various test substances. Experiments performed in spontaneously hypertensive rats (SHR) are supplied by Taconic Farms, Germantown, N.Y. (Tac:N(SHR)_{f}BR). A radiotelemetric device (Data Sciences. International, Inc., St. Paul, Minn.) is implanted into the lower abdominal aorta of all test animals between the ages of 14 to 16 weeks of age. All SHR are allowed to recover from the surgical implantation procedure for at least 2 weeks prior to the initiation of the experiments. Cardiovascular parameters are continuously monitored via the radiotransmitter and transmitted to a receiver where the digitized signal is then collected and stored using a computerized data acquisition system. Blood pressure (mean arterial, systolic and diastolic pressure) and heart rate are monitored in conscious, freely moving and undisturbed SHR in their home cages. The arterial blood pressure and heart rate are measured every 10 min for 10 seconds and recorded. Data reported for each rat represent the mean values averaged over a 24 hour period and are made up of the 144-10 min samples collected each day. The baseline values for blood pressure and heart rate consist of the average of three consecutive 24 hour readings taken prior to initiating the drug treatments. All rats are individually housed in a temperature and humidity controlled room and are maintained on a 12 hour light dark cycle.

In addition to the cardiovascular parameters, weekly determinations of body weight also are recorded in all rats. Treatments are administered in the drinking water, via daily oral gavage or in osmotic minipumps as stated above. If given in drinking water, water consumption is measured five times per week. Doses of the active agents for individual rats are then calculated based on water consumption for each rat, the concentration of drug substance in the drinking water, and individual body weights. All drug solutions in the drinking water are made up fresh every three to four days.

Upon completion of the chronic studies, SHR or DOCA-salt rats are anesthetized and the heart rapidly removed. After separation and removal of the atrial appendages, left ventricle and left plus right ventricle (total) are weighed and recorded. Left ventricular and total ventricular mass are then normalized to body weight and reported. All values reported for blood pressure and cardiac mass represent the group mean ±sem.

Vascular function and structure are evaluated after treatment to assess the beneficial effects of the combination. SHR are studied according to the methods described by Intengan H D, Thibault G, Li J S, Schiffrin E L, Circulation 100 (22): 2267-2275, 1999. Similarly, the methodology for assessing vascular function in DOCA-salt rats is described in Intengan H D, Park J B, Schiffrin, E L, Hypertension 34 (4 Part 2): 907-913, 1999.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative of certain aspects of the present invention and are not a limitation of the scope of the present invention in any way.

### EXAMPLES

The products or the combinations described in the examples below provide medicaments with unexpected therapeutic benefits, or superior or more efficient properties to those of individual monotherapies.

### Example 1

Composition of aliskiren 150 mg (free base) uncoated tablets in mg/unit.

| | Roller | | | |
|---|---|---|---|---|
| | compacted | Dosage | Dosage | Dosage |
| Component | tablet | form 1 | form 2 | form 3 |
| Aliskiren hemi-fumarate | 165.750 | 165.750 | 165.750 | 165.750 |
| Microcrystalline cellulose | 220.650 | 84.750 | 72.250 | 107.250 |
| Polyvinylpyrrolidon K 30 | — | — | 12.000 | 12.000 |
| Crospovidone | 84.000 | 45.000 | 44.000 | 48.200 |
| Aerosil 200 | 4.800 | 1.500 | 1.500 | 1.800 |
| Magnesium stearate | 4.800 | 3.000 | 4.500 | 5.000 |
| Total weight | 480.000 | 300.000 | 300.000 | 340.000 |

Composition of aliskiren 150 mg (free base) uncoated tablets in % by weight.

| | Roller | | | |
|---|---|---|---|---|
| | compacted | Dosage | Dosage | Dosage |
| Component | tablet | form 1 | form 2 | form 3 |
| Aliskiren hemi-fumarate | 34.53 | 55.25 | 55.25 | 48.75 |
| Microcrystalline cellulose | 45.97 | 28.25 | 24.08 | 31.545 |
| Polyvinylpyrrolidon K 30 | — | — | 4 | 3.53 |
| Crospovidone | 17.5 | 15 | 14.67 | 14.175 |
| Aerosil 200 | 1 | 0.5 | 0.5 | 0.53 |
| Magnesium stearate | 1 | 1 | 1.5 | 1.47 |
| Total % | 100.00 | 100.00 | 100.00 | 100.00 |

Composition of aliskiren 150 mg (free base) uncoated tablets in mg/unit (divided into inner/outer phase).

| | | Roller | | | |
|---|---|---|---|---|---|
| | | compacted | | | |
| | Component | tablet | Dosage form 1 | Dosage form 2 | Dosage form 3 |
| Inner | Aliskiren hemi-fumarate | 165.75 | 165.75 | 165.75 | 165.75 |
| Phase | Microcrystalline cellulose | 220.65 | 84.75 | 72.25 | 90.25 |
| | Polyvinylpyrrolidon K 30 | — | — | 12.00 | 12.00 |
| | Crospovidone | 36.00 | — | — | 14.20 |
| | Aerosil 200 | — | — | — | — |
| | Magnesium stearate | 2.40 | — | — | — |
| Outer | Crospovidone | 48.00 | 45.00 | 44.00 | 34.00 |
| phase | Microcrystalline cellulose | — | — | — | 17.00 |
| | Aerosil 200 | 4.80 | 1.50 | 1.50 | 1.80 |
| | Magnesium stearate | 2.40 | 3.00 | 4.50 | 5.00 |
| | Total weight | 480.00 | 300.00 | 300.00 | 340.00 |

Composition of aliskiren 150 mg (free base) uncoated tablets in % by weight (divided into inner/outer phase).

| | | Roller | | | |
|---|---|---|---|---|---|
| | | compacted | | | |
| | Component | tablet | Dosage form 1 | Dosage form 2 | Dosage form 3 |
| Inner | Aliskiren hemi-fumarate | 34.53 | 55.25 | 55.25 | 48.75 |
| Phase | Microcrystalline cellulose | 45.97 | 28.25 | 24.08 | 26.545 |
| | Polyvinylpyrrolidon K 30 | — | — | 4 | 3.530 |
| | Crospovidone | 7.5 | — | — | 4.175 |
| | Aerosil 200 | — | — | — | — |
| | Magnesium stearate | 0.5 | — | — | — |
| Outer | Crospovidone | 10 | 15 | 14.67 | 10 |
| phase | Microcrystalline cellulose | — | — | — | 5 |
| | Aerosil 200 | 1 | 0.5 | 0.5 | 0.53 |
| | Magnesium stearate | 0.5 | 1 | 1.5 | 1.47 |
| | Total % | 100.00 | 100.00 | 100.00 | 100.00 |

### Example 2

Composition of aliskiren (dosage form 3) film-coated tablets in mg/unit.

| | Dosage form 3/Strength | | |
|---|---|---|---|
| | 75 mg | 150 mg | 300 mg |
| Component | (free base) | (free base) | (free base) |
| Aliskiren hemi-fumarate | 82.875 | 165.750 | 331.500 |
| Microcrystalline cellulose | 53.625 | 107.250 | 214.500 |
| Polyvinylpyrrolidon K 30 | 6.000 | 12.000 | 24.000 |
| Crospovidone | 24.100 | 48.200 | 96.400 |
| Aerosil 200 | 0.900 | 1.800 | 3.600 |
| Magnesium stearate | 2.500 | 5.000 | 10.000 |
| Total tablet weight | 170.000 | 340.000 | 680.000 |
| Opadry premix white | 9.946 | 16.711 | 23.9616 |
| Opadry premix red | 0.024 | 0.238 | 1.8382 |
| Opadry premix black | 0.030 | 0.051 | 0.2002 |
| Total fim-coated tablet | 180.000 | 357.000 | 706.000 |
| weight | | | |

The dosages forms 1, 2 and 3 may be prepared, e.g., as follows:
1) mixing the active ingredient and additives and granulating said components with a granulation liquid;
2) drying a resulting granulate;
3) mixing the dried granulate with outer phase excipients;
4) compressing a resulting mixture to form a solid oral dosage as a core tablet; and
5) optionally coating a resulting core tablet to give a film-coated tablet.

The granulation liquid can be ethanol, a mixture of ethanol and water, a mixture of ethanol, water and isopropanol, or a solution of polyvinylpyrrolidones (PVP) in the before mentioned mixtures. A preferred mixture of ethanol and water ranges from about 50/50 to about 99/1 (% w/w), most preferrably it is about 94/6 (% w/w). A preferred mixture of ethanol, water and isopropanol ranges from about 45/45/5to about 98/1/1 (% w/w/w), most preferably from about 88.5/5.5/6.0 to about 91.5/4.5/4.0 (% w/w/w). A preferred concentration of PVP in the above named mixtures ranges from about 5 to about 30% by weight, preferably from about 15 to about 25%, more preferably from about 16 to about 22%.

Attention is drawn to the numerous known methods of granulating, drying and mixing employed in the art, e.g., spray granulation in a fluidized bed, wet granulation in a high-shear mixer, melt granulation, drying in a fluidized-bed dryer, mixing in a free-fall or tumble blender, compressing into tablets on a single-punch or rotary tablet press.

The manufacturing of the granulate can be performed on standard equipment suitable for organic granulation processes. The manufacturing of the final blend and the compression of tablets can also be performed on standard equipment.

For example, step (1) may be carried out by a high-shear granulator, e.g., Collette Gral; step (2) may be conducted in a fluid-bed dryer; step (3) may be carried out by a free-fall mixer (e.g. container blender, tumble blender); and step (4) may be carried out using a dry compression method, e.g., a rotary tablet press.

### Example 3

### Film-Coated Tablets

| | Composition | |
|---|---|---|
| Components | Per Unit (mg) | Standards |
| Granulation | | |
| Valsartan [=active ingredient] | 80.00 | |
| Microcrystalline cellulose/ | 54.00 | NF, Ph. Eur |
| Avicel PH 102 | | |
| Crospovidone | 20.00 | NF, Ph. Eur |
| Colloidal anhydrous silica/ | 0.75 | Ph. Eur/NF |
| colloidal silicon dioxide/Aerosil 200 | | |
| Magnesium stearate | 2.5 | NF, Ph. Eur |
| Blending | | |
| Colloidal anhydrous silica/ | 0.75 | Ph. Eur/NF |
| colloidal silicon dioxide/Aerosil 200 | | |
| Magnesium stearate | 2.00 | NF, Ph. Eur |
| Coating | | |
| Purified water*⁾ | — | |
| DIOLACK pale red 00F34899 | 7.00 | |
| Total tablet mass | 167.00 | |

*⁾Removed during processing.

The film-coated tablets may be manufactured, e.g., as follows:

A mixture of valsartan, microcrystalline cellulose, crospovidone, part of the colloidal anhydrous silica/colloidal silicon dioxide/Aerosile 200, silicon dioxide and magnesium stearate is premixed in a diffusion mixer and then sieve through a screening mill. The resulting mixture is again pre-mixed in a diffusion mixer, compacted in a roller compactor and then sieve through a screening mill. To the resulting mixture, the rest of the colloidal anhydrous silica/colloidal silicon dioxide/Aerosile 200 are added and the final blend is made in a diffusion mixer. The whole mixture is compressed in a rotary tabletting machine and the tablets are coated with a film by using Diolack pale red in a perforated pan.

### Example 4

### Film-Coated Tablets

| | Composition | |
|---|---|---|
| Components | Per Unit (mg) | Standards |
| Granulation | | |
| Valsartan [=active ingredient] | 160.00 | |
| Microcrystalline cellulose/ | 108.00 | NF, Ph. Eur |
| Avicel PH 102 | | |
| Crospovidone | 40.00 | NF, Ph. Eur |
| Colloidal anhydrous silica/ | 1.50 | Ph. Eur/NF |
| colloidal silicon dioxide/Aerosil 200 | | |
| Magnesium stearate | 5.00 | NF, Ph. Eur |
| Blending | | |
| Colloidal anhydrous silica/ | 1.50 | Ph. Eur/NF |
| colloidal silicon dioxide/Aerosil 200 | | |
| Magnesium stearate | 4.00 | NF, Ph. Eur |
| Coating | | |
| Opadry Light Brown 00F33172 | 10.00 | |
| Total tablet mass | 330.00 | |

The film-coated tablets are manufactured, e.g., as described in Example 3.

### Example 5

### Film-Coated Tablets

| | Composition | |
|---|---|---|
| Components | Per Unit (mg) | Standards |
| Core: Internal phase | | |
| Valsartan | 40.00 | |
| [=active ingredient] | | |
| Silica, colloidal anhydrous | 1.00 | Ph. Eur, USP/NF |
| (Colloidal silicon dioxide) | | |
| [=Glidant] | | |
| Magnesium stearate | 2.00 | USP/NF |
| [=Lubricant] | | |
| Crospovidone | 20.00 | Ph. Eur |
| [Disintegrant] | | |
| Microcrystalline cellulose | 124.00 | USP/NF |
| [=Binding agent] | | |
| External phase | | |
| Silica, colloidal anhydrous, | 1.00 | Ph. Eur, USP/NF |
| (Colloidal silicon dioxide) | | |
| [=Glidant] | | |
| Magnesium stearate | 2.00 | USP/NF |
| [Lubricant] | | |
| Film coating | | |
| Opadry ® brown OOF 16711*⁾ | 9.40 | |
| Purified Water**⁾ | — | |
| Total tablet mass | 199.44 | |

*⁾The composition of the Opadry ® brown OOF16711 coloring agent is tabulated below.
**⁾Removed during processing.

Opadry® Composition:

| | Approximate % |
|---|---|
| Ingredient | Composition |
| Iron oxide, black (C.I. No. 77499, E 172) | 0.50 |
| Iron oxide, brown (C.I. No. 77499, E 172 | 0.50 |
| Iron oxide, red (C.I. No. 77491, E 172) | 0.50 |
| Iron oxide, yellow (C.I. No. 77492, E 172) | 0.50 |
| Macrogolum (Ph. Eur) | 4.00 |
| Titanium dioxide (C.I. No. 77891, E 171) | 14.00 |
| Hypromellose (Ph. Eur) | 80.00 |

The film-coated tablets are manufactured, e.g., as described in Example 3.

### Example 6

### Capsules

| Components | Composition Per Unit (mg) |
|---|---|
| Valsartan [=active ingredient] | 80.00 |
| Microcrystalline cellulose | 25.10 |
| Crospovidone | 13.00 |
| Povidone | 12.50 |
| Magnesium stearate | 1.30 |
| Sodium lauryl sulphate | 0.60 |
| Shell | |
| Iron oxide, red | 0.123 |
| (C.I. No. 77491, EC No. E 172) | |
| Iron oxide, yellow | 0.123 |
| (C.I. No. 77492, EC No. E 172) | |
| Iron oxide, black | 0.245 |
| (C.I. No. 77499, EC No. E 172) | |
| Titanium dioxide | 1.540 |
| Gelatin | 74.969 |
| Total mass | 209.50 |

The capsules may be manufactured, e.g., as follows:

### Granulation/Drying:

Valsartan and microcrystallin cellulose are spray-granulated in a fluidized bed granulator with a granulating solution consisting of povidone and sodium lauryl sulphate dissolved in purified water. The granulate obtained is dried in a fluidized bed dryer.

### Milling/Blending:

The dried granulate is milled together with crospovidone and magnesium stearate. The mass is then blended in a conical srew type mixer for approximately 10 minutes.

### Encapsulation:

The empty hard gelatin capsules are filled with the blended bulk granules under controlled temperature and humidity conditions. The filed capsules are dedusted, visually inspected, weight checked and quarantined until by Quality assurance department.

### Example 7

### Capsules

| Components | Composition Per Unit (mg) |
|---|---|
| Valsartan [=active ingredient] | 160.00 |
| Microcrystalline cellulose | 50.20 |
| Crospovidone | 26.00 |
| Povidone | 25.00 |
| Magnesium stearate | 2.60 |
| Sodium lauryl sulphate | 1.20 |
| Shell | |
| Iron oxide, red | 0.123 |
| (C.I. No. 77491, EC No. E 172) | |
| Iron oxide, yellow | 0.123 |
| (C.I. No. 77492, EC No. E 172) | |
| Iron oxide, black | 0.245 |
| (C.I. No. 77499, EC No. E 172) | |
| Titanium dioxide | 1.540 |
| Gelatin | 74.969 |
| Total mass | 342.00 |

The capsules are manufactured, e.g., as described in Example 6.

### Example 8

### Hard Gelatine Capsules

| Components | Composition Per Unit (mg) |
|---|---|
| Valsartan [=active ingredient] | 80.00 |
| Sodium laurylsulphate | 0.60 |
| Magnesium stearate | 1.30 |
| Povidone | 12.50 |
| Crospovidone | 13.00 |
| Microcrystalline cellulose | 21.10 |
| Total mass | 130.00 |

### Example 9

### Hard Gelatin Capsules

| Components | Composition Per Unit (mg) |
|---|---|
| Valsartan [=active ingredient] | 80.00 |
| Microcrystalline cellulose | 110.00 |
| Povidone K30 | 45.20 |
| Sodium laurylsulphate | 1.20 |
| Magnesium stearate | 2.60 |
| Crospovidone | 26.00 |
| Total mass | 265.00 |

Components (1) and (2) are granulated with a solution of components (3) and (4) in water. The components (5) and (6) are added to the dry granulate and the mixture is filled into size 1 hard gelatin capsules.

### Example 10

Composition and quantities for a combination of valsartan and amiodipine

| | COMPOSITION PER | |
|---|---|---|
| Components | UNIT (mg) | COMPOSITION (%) |
| Diovan Drug Substance | 80.00 | 43.02 |
| Amlodipine Drug | 6.94 | 3.73 |
| Substance | | |
| Avicel 102 (I) | 54.00 | 29.04 |
| Avicel 102 (II) | 20.00 | 10.76 |
| Crospovidone (I) | 15.00 | 8.07 |
| Crospovidone (II) | 4.0 | 2.15 |
| Cab-O-Sil | 1.50 | 0.81 |
| Magnesium Stearate (I) | 3.00 | 1.61 |
| Magnesium Stearate (II) | 1.50 | 0.81 |
| | 185.94 | 100.00 |

The tablet is manufactured e.g. essentially as described in Formulation Example 1.

All publications and patents mentioned herein are incorporate by reference in their entirety as if set forth in full herein.

1: A combination comprising:
(a). an angiotensin II receptor blocker (ARB), or a pharmaceutically acceptable salt thereof;
(b). a calcium channel blocker (CCB), or a pharmaceutically acceptable salt thereof; and
(c). one of the two active agents selected from
(i). a renin inhibitor, or a pharmaceutically acceptable salt thereof;
(ii). a neutral endopeptidase (NEP) inhibitor, or a pharmaceutically acceptable salt thereof.

2. The combination according to claim 1, wherein the angiotensin II receptor is valsartan, or a pharmaceutically acceptable salt thereof.

3. The combination according to claim 1 wherein the calcium channel blocker is amlodipine, or a pharmaceutically acceptable salt thereof.

4. The combination according to claim 1, wherein a renin inhibitor is selected from the group consisting of RO 66-1132, RO 66-1168 and a compound of the formula 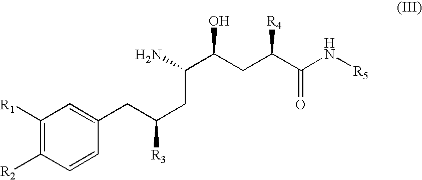
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

5. The combination according to claim 4, wherein a renin inhibitor is a compound of formula (III) having the formula 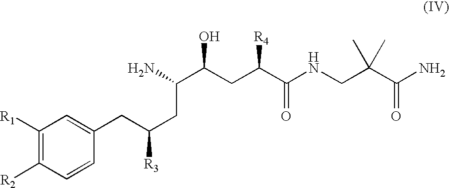
wherein R₁ is 3-methoxypropyloxy; R₂ is methoxy; and R₃ and R₄ are isopropyl; or a pharmaceutically acceptable salt thereof.

6. The combination according to claim 5, wherein the compound of formula (IV) is in the form of the hemi-fumarate salt thereof.

7. The combination according to claim 1, wherein a neutral endopeptidase inhibitor is selected from the group consisting of SQ 28,603, N—[N-[1(S)-carboxyl-3-phenylproplyl]-(S)-phenylalanyl]-(S)-isoserine, N—[N-[((1S)-carboxy-2-phenyl)ethyl]-(S)-phenylalanyl]-β-alanine, N-[2(S)-mercaptomethyl-3-(2-methylphenyl)-propionyl]methionine, (cis-4-[[[1-[2-carboxy-3-(2-methoxyethoxy)propyl]-cyclopentyl]carbonyl]amino]-cyclohexane-carboxylic acid), thiorphan, retro-thiorphan, phosphoramidon, SQ 29,072, N-(3-carboxy-1-oxopropyl)-(4S)-p-phenyl-phenylmethyl)-4-amino-(2R)-methylbutanoic acid ethyl ester, (S)-cis-4-[1-[2-(5-indanyloxy-carbonyl)-3-(2-methoxyethoxy)propyl]-1-cyclopentanecarboxamido]-1-cyclohexanecarboxylic acid, 3-(1-[6-endo-hydroxymethylbicyclo[2,2,1]heptane-2-exo-carbamoyl]cyclopentyl)-2-(2-methoxyethyl)propanoic acid, N-(1-(3-(N-t-butoxycarbonyl-(S)-prolylamino)-2(S)-t-butoxy-carbonylpropyl)cyclopentanecarbonyl)-O-benzyl-(S)-serine methyl ester, 4-[[2-(mercapto-methyl)-1-oxo-3-phenylpropyl]amino]benzoic acid, 3-[1-(cis-4-carboxycarbonyl-cis-3-butylcyclohexyl-r-1-carboamoyl)cyclopentyl]-2S-(2-methoxyethoxy-methyl)propanoic acid, N-((2S)-2-(4-biphenylmethyl)-4-carboxy-5-phenoxyvaleryl)glycine, N-(1-(N-hydroxycarbamoyl-methyl)-1-cyclopentanecarbonyl)-L-phenylalanine, (S)-(2-biphenyl-4-yl)-1-(1H-tetrazol-5-yl)ethylamino)methylphosphonic acid, (S)-5-(N-(2-(phosphonomethyl-amino)-3-(4-biphenyl)-propionyl)-2-aminoethyl)tetrazole, β-Alanine, 3-[1,1′-biphenyl]-4-yl-N-[diphenoxyphosphinyl)-methyl]-L-alanyl, N-(2-carboxy-4-thienyl)-3-mercapto-2-benzylpropanamide, 2-(2-mercapto-methyl-3-phenylpropionamido)thiazol-4-ylcarboxylic acid, (L)-(1-((2,2-dimethyl-1,3-dioxolan-4-yl)-methoxy)carbonyl)-2-phenylethyl)-L-phenylalanyl)-β-alanine, N—[N-[(L)-[1-[(2,2-dimethyl-1,3-dioxolan-4-yl)-methoxy]carbonyl]-2-phenylethyl]-L-phenylalanyl]-(R)-alanine, N-[-N-[(L)-1-carboxy-2-phenylethyl]-L-phenylalanyl]-(R)-alanine, N-[2-acetylthiomethyl-3-(2-methyl-phenyl)propionyl]-methionine ethyl ester, N-[2-mercapto-methyl-3-(2-methylphenyl)-propioyl]-methionine, N-[2(S)-mercaptomethyl-3-(2-methylphenyl)propanoyl]-(S)-isoserine, N—(S)-[3-mercapto-2-(2-methylphenyl)propionyl]-(S)-2-methoxy-(R)-alanine, N-[1-[[1(S)-benzyloxycarbonyl-3-phenylpropyl]amino]-cyclopentylcarbonyl]-(S)-isoserine, N-[1-[[1(S)-carbonyl-3-phenylpropy]amino]-cyclopentylcarbonyl]-(S)-isoserine, 1,1′-[dithiobis-[2(S)-(2-methylbenzyl)-1-oxo-3,1-propanediyl]]-bis-(S)-isoserine, 1,1′-[dithiobis-[2(S)-(2-methylbenzyl)-1-oxo-3,1-propanediyl]]-bis-(S)-methionine, N-(3-phenyl-2-(mercaptomethyl)-propionyl)-(S)-4-(methylmercapto)-methionine, N-[2-acetylthiomethyl-3-phenyl-propionyl]-3-aminobenzoic acid, N-[2-mercapto-methyl-3-phenyl-propionyl]-3-aminobenzoic acid, N-[1-(2-carboxy-4-phenylbutyl)-cyclopentanecarbonyl]-(S)-isoserine, N-[1-(acetylthiomethyl)-cyclopentane-carbonyl]-(S)-methionine ethyl ester, 3(S)-[2-(acetylthiomethyl)-3-phenyl-propionyl]amino-ε-caprolactam and N-(2-acetylthiomethyl-3-(2-methylphenyl)propionyl)-methionine ethyl ester, or in each case, a pharmaceutically acceptable salt thereof.

8. The combination according to, claim 1 wherein a neutral endopeptidase inhibitor is N-(3-carboxy-1-oxopropyl)-(4S)-p-phenylphenylmethyl)-4-amino-(2R)-methylbutanoic acid ethyl ester, or a pharmaceutically acceptable salt thereof; or N-(3-carboxy-1-oxopropyl)-(4S)-p-phenylphenylmethyl)-4-amino-(2R)-methylbutanoic acid, or a pharmaceutically acceptable salt thereof.

9. The combination according to, claim 1 further containing a diuretic.

10. The combination according to claim 9, wherein the diuretic is hydrochlorothiazide, or a pharmaceutically acceptable salt thereof.

11. A pharmaceutical composition comprising the combination according to claim 1 and a pharmaceutically acceptable carrier.

12. The pharmaceutical composition according to claim 11 for the prevention of, delay the onset of and/or treatment of cardiovascular disorders.

13. The pharmaceutical composition according to claim 12, wherein the of cardiovascular disorder is selected from the group consisting of hypertension, heart failure, left ventricular dysfunction, endothelial dysfunction, diastolic dysfunction, hypertrophic cardiomyopathy, diabetic cardiac myopathy, supraventricular and ventricular arrhythmias, atrial fibrillation, cardiac fibrosis, atrial flutter, detrimental vascular remodeling, plaque stabilization, myocardial infarction and its sequelae, atherosclerosis, angina pectoris, renal insufficiency, renal fibrosis, polycystic kidney disease, type 2 diabetes, metabolic syndrome, secondary aldosteronism, primary and secondary pulmonary hypertension, nephrotic syndrome, diabetic nephropathy, glomerulonephritis, scleroderma, glomerular sclerosis, proteinuria of primary renal disease, renal vascular hypertension, diabetic retinopathy, end-stage renal disease, migraine, peripheral vascular disease, Raynaud's disease, luminal hyperplasia, cognitive dysfunction, glaucoma and cerebrovascular disease.

14. A method for the prevention of, delay the onset of and/or treatment of cardiovascular disorders, which method comprises administering to a patient, in need thereof, a therapeutically effective amount of the combination according to claim 1 and a pharmaceutically acceptable carrier.

15. The method according to claim 14, wherein a cardiovascular disorder is selected from the group consisting of hypertension, heart failure, left ventricular dysfunction, endothelial dysfunction, diastolic dysfunction, hypertrophic cardiomyopathy, diabetic cardiac myopathy, supraventricular and ventricular arrhythmias, atrial fibrillation, cardiac fibrosis, atrial flutter, detrimental vascular remodeling, plaque stabilization, myocardial infarction and its sequelae, atherosclerosis, angina pectoris, renal insufficiency, renal fibrosis, polycystic kidney disease, type 2 diabetes, metabolic syndrome, secondary aldosteronism, primary and secondary pulmonary hypertension, nephrotic syndrome, diabetic nephropathy, glomerulonephritis, scleroderma, glomerular sclerosis, proteinuria of primary renal disease, renal vascular hypertension, diabetic retinopathy, end-stage renal disease, migraine, peripheral vascular disease, Raynaud's disease, luminal hyperplasia, cognitive dysfunction, glaucoma and cerebrovascular disease.

16. (canceled)

17. (canceled)
